(19) United States
(12) Patent Application Publication
(10) Pub. No.: US 2024/0415147 A1
(43) Pub. Date: Dec. 19, 2024

(54) FREEZE STRUCTURED AND ENZYMATICALLY CROSSLINKED FOOD MATERIALS

(71) Applicant: Planted Foods AG, Kemptthal (CH)

(72) Inventors: Patrick Ruhs, Zurich (CH); Michael Muller, Uster (CH); Laura Alessia Savorani, Zurich (CH)

(21) Appl. No.: 18/700,150

(22) PCT Filed: Oct. 12, 2022
(86) PCT No.: PCT/EP2022/078369

§ 371 Date: Apr. 10, 2024

(30) Foreign Application Priority Data
Oct. 12, 2021 (EP) 21202267.7

(51) Int. Cl. A23J 3/24; A23J 3/14; A23J 3/22; C12N 9/10

(52) U.S. Cl. CPC A23J 3/24; A23J 3/14; A23J 3/227; C12N 9/1044; C12Y 203/02013

(57) ABSTRACT

A method for preparing a crosslinked texturized food stuff, comprising the steps of: providing a food stuff mixture comprising at least a protein, water and a crosslinking enzyme; directionally freezing said food stuff mixture by cooling to below the freezing temperature of the food stuff mixture according to a directional thermal gradient such that ice crystals are grown anisotropically within the food stuff mixture to create a frozen texturized food stuff mixture; and increasing the temperature of said frozen texturized food stuff mixture to a temperature at which the crosslinking enzyme is active and allowing the texturized food stuff mixture to form a crosslinked texturized food stuff.

### RELATED APPLICATIONS

This application is a national phase application claiming priority to International Application PCT/EP2022/078369 filed Oct. 12, 2022, which claims priority to European Application No. 21202267.7, filed Oct. 12, 2021, all of which applications are herein incorporated by reference.

### BACKGROUND

The invention relates to freeze structured and enzymatically crosslinked materials, more specifically food materials, processes for the production thereof, and the products created from said processes. The invention aims to provide meat or fish analogue products from non-animal based protein sources for human consumption.

In the last decades, much research has focused on the challenge to provide sufficient food for an increasing human population. Popular protein sources such as meat from animals have the disadvantage that their production is highly energy inefficient and due to their massive scale have a big impact on the global environment through the emission of greenhouse gases and high water usage. Furthermore, the consumption and production of animal protein also raises ethical concerns in a growing sector of the human populous. Hence, a shift towards food products generated from non-animal proteins that can substitute meat would make a big contribution towards fulfilling the protein requirements for the growing population. To improve the acceptance in a large customer segment accustomed to consuming meat, non-animal protein-based food products have to have texture and mouthfeel replicating the ones found in animal meat products.

The introduction of textures into food products from non-animal proteins for meat or fish analogues has been achieved by others through various processes such as extrusion, shear cell technology or freeze structuring. Freeze structuring, also known as freeze casting or ice-templating, is commonly employed to create porous, anisotropic structures made from engineering materials such as ceramics, metals or polymers (Sylvain Deville, Freezing Colloids: Observations, Principles, Control, and Use, Springer Press, 2017). The process involves the creation of a stable particle suspension or a slurry which is then subjected to temperatures at which the dispersing fluid (usually water) freezes. The freezing can occur from one or several sides of the dispersion (G. Shao et al., Adv. Mater. 2020, 32, 1907176) which, in the case of water, leads to the formation of anisotropic ice crystals perpendicular to the freezing surface. These ice crystals push the dispersed solid phase aside to form areas of accumulated particles. In the case of food products, these particles comprise protein powders and edible carbohydrates. The texturized food products created during the freeze structuring process are then required to be stabilized, commonly either by employing heat, freeze-drying, gelling agents or non-solvents. Without the stabilization process, the melting of the ice crystals can potentially lead to the partial or complete loss of texture, depending on the concentration and type of solids. In other cases, the lack of a stabilization step leads to very brittle texturized food products which do not resemble meat or fish upon consumption. Hence, the stabilization process can be considered a crucial step in the creation of freeze structured food products.

In U.S. Pat. No. 4,084,017A, texturized protein materials are prepared through freeze structuring, followed by a shock fracturing treatment where the frozen mass is subjected to temperatures of at least 50° C. lower than its current temperature, to induce fractures at the weak planes and thereby further texturize the food product. The protein is then stabilized, which is achieved through freeze drying or submersion into a non-solvent, ethanol, and subsequent heating to gel the protein. While the utilized methods, freeze drying, stabilization with ethanol and heating, sufficiently stabilize the created texture, ultimately they partially or fully dehydrate the final product, preventing the final product from being juicy, which is a desired property for meat or fish analogues from non-animal proteins. Furthermore, heat sensitive nutrients could not be included in such a product due to temperatures employed during the protein gelling process. “Juicy” denotes a sensory attribute perceived when eating e.g., fresh fruit and vegetable (water release) or meat (water and oil release), and describes the amount of liquid released during mastication, the force at which the juice is expelled, the amount of juice released at the first bite and over time, the consistency of the juice, and the contrast between liquid and solid. Thus, to be perceived as juicy, a food product is required physically entrap a liquid within its structure, rather than chemically bind it, and release the liquid upon compression or breaking such as during mastication.

Further background art in the area of freeze structuring food products includes EP0037676A2, where stabilization of the freeze structured protein mass is realized by the addition of sodium alginate. The freeze structured protein mass is sliced and exposed to calcium salts to gel the mass. This is followed by heating to gel the protein, subsequent removal of undesirable salts and softening using a sequestering agent. Again, the suggested heat treatments, all of which are above 100° C., can potentially lead to product dehydration and destroy heat sensitive nutrients. Also, the slicing required to enable the gelation of alginate throughout the sample ultimately limits the thickness of texturized foods products that are obtainable.

JPS61187756A employed directional freezing and fixing an aqueous dispersion sol, gel or paste comprising ice-nucleating bacteria to reduce the undercooling required for ice crystal formation. Following the directional freezing, a fixation step was applied to stabilize the structure, selected from either heat fixation, freeze-drying or the application of the chemical crosslinker glutaraldehyde, which is toxic and a strong irritant. Furthermore, the latter does not penetrate deep into the gel or paste due to an immediate crosslinking reaction with the proteins, preventing diffusion and thorough crosslinking of thicker samples.

U.S. Pat. No. 3,870,808A also employed freezing a proteinaceous slurry to create meat simulating textured food, however, freezing was not directional but isotropic and heat setting at a minimum of 150° F. was required to stabilize the final structure. In U.S. Pat. No. 3,920,853A, structured food products were created through freeze structuring and quickly heating the frozen mass to gel the protein and stabilize the structure.

While the presented prior art strives to create structured protein products, the processing steps to stabilize the structured protein mass fail to maintain a high moisture content which is important for juiciness and/or require a final heating step to gel the protein, preventing the incorporation of heat sensitive nutrients and potentially leading to further protein denaturation and subsequent loss of water from the meat or fish analogue. Furthermore, raw materials with proper heat-gelling ability are required, which limits the choice of raw materials, especially for proteins from plant sources, which tend to have limited heat-gelling ability. Also, the heat treatment of the texturized foods requires them to be placed in an appropriate container, as otherwise the high temperature destroys the shape of the texturized food. Moreover, addition of reactive crosslinking agent to a food stuff mixture only after freezing limits the size of texturized food stuff that can be manufactured, as diffusion limitations of the crosslinking agent within the food stuff mixture apply, leading to incomplete and inhomogeneous crosslinking throughout the food stuff mixture.

This need for better solutions is addressed by the presented invention.

### SUMMARY

According to a first aspect, the invention relates to a method for preparing a crosslinked texturized food stuff, said method comprising the following steps:
a) providing a food stuff mixture comprising at least a protein, water and a crosslinking enzyme;
b) directionally freezing said food stuff mixture by cooling to below the freezing temperature of the food stuff mixture according to a directional thermal gradient such that ice crystals are grown anisotropically within the food stuff mixture to create a frozen texturized food stuff mixture;
c) increasing the temperature of said frozen texturized food stuff mixture to a temperature at which the crosslinking enzyme is active and allowing the texturized food stuff mixture to form a crosslinked texturized food stuff.

The presence of the crosslinking enzyme in the food stuff mixture before directional freezing can ensure that crosslinking occurs homogeneously throughout the entire foodstuff mixture and the process of the herein presented invention is therefore not subject to diffusion limitations of the crosslinker from a surrounding solution into the foodstuff mixture.

As will be understood by those skilled in the art, the method may comprise, in particular, a combination of at least a two separate process steps which include the freeze texturizing and subsequent crosslinking of the food stuff mixture.

Optionally, the ice crystals are elongated. “Elongated” for the purpose of this invention means that the ice crystals are longer in one or two of the three dimensions, with “longer” meaning that the aspect ratio of one or two of the three dimensions is at least a factor of 1.5. With the formation of elongated ice crystals, the food stuff mixture is transformed into a texturized food stuff mixture with a lamellar or fibrillar structure templated by the ice crystals.

The formation of the elongated ice crystals simultaneously leads to the concentration of solutes and solids in between the crystals, locally increasing the concentration, and therefore further enabling the crosslinking enzyme to be effective.

Subsequently upon increasing the temperature, the frozen watery phase contained in the frozen texturized food stuff mixture melts and the crosslinking enzymes become active, thus irreversibly stabilizing the lamellar or fibrillar structure through crosslinking and creating a firm food stuff.

As a heat treatment step with temperatures over 50° C. to stabilize the texturized food stuff may be unnecessary when using the crosslinking enzyme, the process is more energy efficient, scalable and enables use of live cultures (e.g. living starter cultures) such as bacteria, fungi, mycelium or spores or native proteins in the food stuff mixture during the process. It also allows the creation of juicy, texturized food stuffs which can contain heat-sensitive nutrients and can be used as meat or fish analogues which have one or more of:
i) a directed, lamellar or fibrillar structure throughout the entire food stuff, and/or
ii) consistencies that resemble meat or fish across the entire food stuff, and/or
iii) high amounts of free water throughout the entire food stuff to provide the right juiciness, and/or
iv) the shape of meat or fish products by directionally freezing the food stuff in a suitable mold.

Optionally, the protein is provided in the food stuff mixture at a concentration of at least 2 wt %, optionally at least 5 wt %, optionally at least 8 wt. %, optionally at least 12 wt %, and/or wherein said protein is not derived from animal slaughter or animal farming, or said protein is derived from plants, fungi, algae, bacteria or cell cultures.

Optionally, the pH of the food stuff mixture is adjusted between 4 and 9, optionally wherein the pH adjustment is performed through the addition of food grade acids and bases.

Optionally, the food stuff mixture further comprises living starter cultures (microorganisms), selected from the group of bacteria, fungi or fungal spores.

The crosslinking enzyme may be selected from the group consisting of transglutaminase, sortases, lysyl oxidases and amine oxidases, tyrosinases, laccases or peroxidases forming ε-(γ-glutamyl) lysine bridges, an LPXTG motif bridge, a Schiff-Base or aldol condensation products, o-quinone or ditryrosyl-type crosslinks, respectively.

As it is clear to someone skilled in the food processing industry crosslinking enzymes are commonly provided immobilized within or onto a carrier matrix. The crosslinking enzymes may, in some configurations of the present invention, be within a carrier matrix such as maltodextrin.

The amount of crosslinking enzyme, given in wt. % relative to comprised protein or food stuff mixture can also be directly translated into enzyme activity units [U/g] relative to the comprised protein or the overall food stuff mixture. Hence, the final enzyme activity units in the food stuff mixture is calculated using the specific enzyme activity units (given in [Uₛ/g]) of the immobilized crosslinking enzyme and the amounts added. A specified amount of (x) wt. % of crosslinking enzyme relative to protein or food stuff mixture, translates to into enzyme activity units [U] relative to 100 g of the comprised protein or the overall food stuff mixture by multiplying (x) wt. % with the specific enzyme activity units.

Optionally, the crosslinking enzyme added to the food stuff mixture has a specific enzyme activity (given in activity units, Uₛ/g) of 60-150 Uₛ/g, optionally of 80-135 Uₛ/g, optionally of 90-125 Uₛ/g.

Optionally, the crosslinking enzyme may be added to the food stuff mixture at a concentration relative to the comprised protein of at least 0.1 wt. % (9 U/100 g of protein), optionally at least 0.5 wt. % (45 U/100 g of protein), more preferably at least 1 wt. % (90 U/100 g of protein) and/or up to 5 wt. % (450 U/100 g of protein), preferably up to 2.5 wt. % (225 U/100 g of protein).

Adjusting protein source or type, protein concentration and enzyme concentration allow to tailor the texture, such as firmness and springiness (i.e. elasticity), by changing the number of crosslinks being formed and the crosslinking kinetics.

The crosslinking enzyme may be active from temperatures of at least 10° C., optionally at least 4° C., optionally at least 2° C. Optionally, said crosslinking enzyme is still active above 20° C. and/or the crosslinking enzyme is active at or below 70° C., optionally at or below 60° C., optionally at or below 50° C.

Optionally, step c) is carried out for at least 5 min, optionally at least 10 min, optionally at least 20 min, optionally at least 30 min.

It is possible that in step c) the temperature is kept at a temperature above the melting point of the frozen liquid contained within the frozen texturized food stuff mixture, optionally above 0° C. and below the deactivation temperature of the enzyme, optionally at a temperature at which at least 50% of the maximum enzyme activity is reached. The method steps b) and c) may be repeated at least once.

It is also envisaged that the food stuff mixture may be shaped into a predefined form through casting, pressing, extrusion or 3D printing.

The directional freezing is optionally performed at temperatures within the range of 0° C. to −80° C., optionally within the range of −4° C. to −30° C., optionally within the range of −10° to −20° C.

Optionally, the method further comprises a step of
d) deactivating the crosslinking enzyme, optionally by heating the crosslinked texturized food stuff to at least the deactivation temperature of the crosslinking enzyme; and/or
e) pressing the crosslinked texturized food stuff to remove water and/or to increase the protein concentration;
f) fermenting whole or partial pieces of the crosslinked texturized food stuff using bacteria and/or fungi, wherein at least one fungus is selected from the group consisting of ascomycetes, basidiomycetes, deuteromycetes, oomycetes, and/or zygomycetes; and/org
g) Cutting or slicing the crosslinked texturized food stuff randomly, optionally along or perpendicular to the fiber direction.

According to a further aspect, the invention relates to a crosslinked texturized food stuff comprising protein, water and at least residues of a crosslinking enzyme, optionally inactive residues of a crosslinking enzyme. Optionally, the crosslinked texturized foodstuff is provided in accordance with the method according to an embodiment of the first aspect of the present invention.

Optionally, at least some of the protein in the food product or in the crosslinked texturized food stuff according to any of the above-mentioned aspects is crosslinked, comprising at least one ε-(γ-glutamyl) lysine bridge, at least one LPXTG motif bridge, or at least one Schiff-Base or aldol condensation product or at least one o-quinone crosslink or at least one ditryrosyl-type crosslink.

The food product or the crosslinked texturized food stuff according any of the above-mentioned aspects optionally has a lamellar or fibrillar structure. The food product or the crosslinked texturized food stuff optionally has a Young's modulus of at least 1 kPa measured in uniaxial compression.

The crosslinked texturized food stuff optionally comprises at least 2 wt %, optionally at least 5 wt %, optionally at least 8%, optionally at least 12 wt % protein content. The product may further comprise lipids and/or fibers from a plant, fungi, single cell or fermented source. The protein may originate from sources other than animal slaughter or animal farming.

In a further aspect of the invention, the crosslinked texturized food stuff may further comprise living starter cultures such as bacteria, fungi, spores or the products originating from such microorganisms such as texturized material based on mycelium. Optionally, the crosslinked texturized food stuff has a water content of at least 80% of the water content of the food stuff mixture, optionally more than 90%, optionally more than 95%.

Optionally, the crosslinked texturized food stuff has an elastic or Young's modulus between 1 kPa and 5 MPa, optionally between 10 kPa and 2 MPa measured in uniaxial compression.

In another aspect of this invention, the crosslinked, texturized food stuff is further exposed to microorganisms such as bacteria or fungi to perform a fermentation step in which the microorganism consume, transform or add matter in and/or on the crosslinked, texturized food stuff to improve taste, juiciness, nutritional value or to increase the mechanical properties or texture.

In another aspect of this invention, the crosslinked, texturized food stuff is further covered, mixed or assembled with mycelium in the form of texturized material or a sheet.

According to a further aspect, the invention relates to a food product resulting from an embodiment of the method described herein.

### BRIEF DESCRIPTION OF FIGURES

The invention is further illustrated by the following examples and figures, from which further embodiments and advantages can be drawn. These examples are meant to illustrate the invention but not to limit its scope.

FIG. 1 depicts a crosslinked texturized food stuff according to the present invention.

FIG. 2a depicts a crosslinked texturized food stuff according to the present invention and containing transglutaminase.

FIG. 2b depicts, for comparison, a similar composition that lacks transglutaminase.

FIG. 3a depicts a crosslinked texturized food stuff according to the present invention after breaking the texturized food stuff parallel to the short axis.

FIG. 3b depicts a crosslinked texturized food stuff according to the present invention after breaking the texturized food stuff perpendicular to the short axis.

FIG. 4a shows a crosslinked texturized food stuff according to the present invention from a top view.

FIG. 4b shows a side view after breaking the cylindrical shape shown in FIG. 4a parallel to the short axis in half.

FIG. 4c depicts fine interlamellar fibers as may be achieved with the present invention as indicated by an arrow.

FIG. 5a shows the different Young's moduli achieved with the two different crosslinking methods compared to a not crosslinked food stuff mixture.

FIG. 5b shows the resulting structures found when looking into the food stuff mixture either after heat treatment (left) or enzymatic treatment (right).

FIG. 6 shows the mechanical response of differently manufactured food stuff mixtures upon 50% strain uniaxial compression, namely a sample that is not freeze structured but crosslinked (No FS, just fridge), freeze structured and crosslinked (FS), and freeze structured but not crosslinked (FS, no tg).

FIG. 7 shows the mechanical response upon 50% strain uniaxial compression of food stuff mixtures crosslinked for either 1 h, 2 h, 3 h or 4 h after freeze structuring.

FIG. 8a shows a counter example of a food stuff mixture upon mixing in a 1% Glutaraldehyde solution, where the fast reaction of the glutaraldehyde with the food stuff mixture leads to a crumbly, non-smooth, and inhomogeneous mixture which is unsuitable for the freeze structuring process.

FIG. 8b shows a side view after breaking the cylindrical shape after attempting to structure the food stuff mixture shown in FIG. 8a. This counter example demonstrates no visible lamella or fibers present and a resulting non-cohesive structure that disintegrated upon compression (as indicated in circled area). This counter example results in a grainy paste rather than a texturized, crosslinked food stuff mixture.

FIG. 9 shows a counter example of the mixing of a food stuff mixture prepared with a 1% glutaraldehyde solution as well as after immersion of the still frozen food stuff mixture in a 10% glutaraldehyde solution. The crosslinked areas in the periphery of the food stuff mixture are darker than the non-crosslinked and crumbly area marked with a dashed line, indicating diffusion limitations of the crosslinker from the outside of the food stuff mixture to its center.

FIG. 10 shows the surface fermentation and colonization by a filamentous fungi of a crosslinked texturized food stuff according to the present invention, forming a skin-like feature on the surface of the crosslinked texturized food stuff.

FIG. 11 shows the resulting structure when a biofilm made from filamentous fungi is included into the food stuff mixture before freezing and crosslinking and the texturized food stuff is pan-fried, where the biofilm in the crosslinked texturized food stuff mixture is clearly seen as thin threads between the lamellae resembling a fat-like or connective tissue-like structure.

### DETAILED DESCRIPTION

To obtain the advantageous properties described above, a provided food stuff mixture comprising protein, water and a crosslinking enzyme is filled in a mold in which one or more surfaces of the food stuff mixture are exposed directly or indirectly (e.g., through the mold) to a cooling medium to freeze the food stuff mixture, resulting in an anisotropic thermal gradient which guides the directional growth of ice crystals along this gradient. This directional freezing or so called “freeze structuring” process results in a frozen texturized food stuff mixture. The food stuff mixture can be given any shape depending on the mold utilized. Optionally, the mold or the food stuff mixture is cooled from a planar surface, with the resulting orientation of the formed elongated ice crystals being perpendicular to the planar surface (i.e. anisotropic growth). In order to form a lamellar or fibrillar structure during freeze-structuring, enzymatic crosslinking prior to freeze-structuring (“premature crosslinking”) optionally is kept at a low level or substantially avoided. This is achieved by short processing times and/or low temperature of the food stuff mixture. Optionally, the food stuff mixture is kept at a temperature of not above 25° C. prior to freezing, more preferably not above 10° C., optionally not above 4° C. to reduce premature crosslinking. Furthermore, the higher the temperature of the food stuff mixture prior to freezing is, the shorter should be the time between enzyme addition and freezing. Optionally, at a temperature of 20° C. to 25° C., the time from mixing enzymes and proteins until freezing the food stuff mixture should be no longer than 30 min. The addition of a crosslinking enzyme before the freezing process is helpful to ensure an even distribution of the enzyme throughout the entire food stuff. The freeze structuring results in a lamellar or fibrillar protein-rich structure. The thickness of the protein-rich lamella or fibers is inversely proportional to the cooling rate employed during the directional freezing process.

When subsequently increasing the temperature of the frozen texturized food stuff mixture, the frozen water starts thawing and when reaching the activation temperature of the enzyme, said enzyme becomes active and crosslinks the proteins throughout the food stuff mixture. The term “active” or “activation temperature” in the present invention with respect to the crosslinking enzyme is defined as that the enzyme is converting a suitable substrate into the desired covalent bonds between proteins, peptides or amino acids. An example of this would be the formation of an isopeptide bond (product) from glutamine and lysin (substrates) using transglutaminase as an enzyme.

The crosslinking process stabilizes the lamellar or fibrillar protein-rich structure, which has been formed during freeze-structuring, to form a crosslinked texturized food stuff with a structure and texture comparable to animal meat or fish flesh. Furthermore, it increases the stiffness of said foodstuff and increases the stability upon heating or cooking, which are both highly important features for meat or fish analogues.

Surprisingly, at lower protein concentrations, successful crosslinking of the food stuff mixture was found to require the combination of the enzymatic crosslinking approach with freeze structuring, as enzymatic crosslinking alone does not lead to a crosslinked food stuff, indicating a surprising enhancing effect of the freeze structuring process on the enzymatic crosslinking.

Surprisingly, the crosslinking process works by simple thawing to room temperature, but can be accelerated by increasing the temperature to where the enzyme shows the highest activity, while maintaining a lamellar or fibrillar structure within the food stuff mixture.

The crosslinked texturized food stuff mixture is subsequently released from the mold and can be further processed.

“Provided” in the above context means the food stuff mixture is either used or prepared. In one embodiment, the food stuff mixture is prepared via a kneading machine, a propeller, spiral, static, planetary or horizontal mixer. The “food stuff mixture” can be in the form of a slurry, paste, foam or an emulsion and comprises at least a protein, a crosslinking enzyme and water.

The term “cooling medium” defines a medium with the capability to directly or indirectly remove heat from the food stuff mixture to lower the temperature of the entire food stuff mixture below the freezing temperature of liquid within the food stuff mixture, optionally water. The medium could be cold gas or a cold liquid, but a cold solid surface, such as a Peltier element is also possible.

The term “mold” hereby is defined as a container which can hold the food stuff mixture and give it a pre-defined shape or simply protect the food stuff mixture from mechanical influences and collapsing under its own weight. The mold can be made, for example, from a plastic or ceramic material alone or from a combination of metal and plastic or ceramic. The mold can partially or completely enclose the food stuff mixture. The mold may consist of one single part (e.g., it may be made from a monolithic material) or it may comprise several separate parts. Furthermore, the mold can also be a soft flexible casing containing and shaping the food stuff mixture and can be made from biological or artificial materials.

Surfaces of the mold which are not insulated from the cooling medium, or surfaces of the mold which have a significantly higher thermal conductivity than other surfaces of the mold, or surfaces of the food stuff mixture which are in direct contact with the cooling medium, determine the thermal gradient, and hence the freezing direction and orientation of the elongated ice crystals in the frozen food stuff mixture. “Insulated” means that there is a material, which has a low thermal conductivity, for example Styrofoam. The material of low thermal conductivity may be provided such that certain portions of the food stuff mixture and/or certain portions of the mold cool down slower than others, thereby providing the thermal gradient. The material of low thermal conductivity may be in direct contact with a surface of the mold, with the food stuff mixture and/or may be part of the mold itself.

Optionally, the freezing takes place from one or more predetermined surfaces of the mold or food stuff mixture. Optionally, the freezing takes place starting from only one predetermined surface of the mold and/or of the food stuff mixture (e.g., from an upper surface of the food stuff mixture that is in contact with the cooling medium). Optionally, the freezing takes place from at least one or only one flat surface of the mold or food stuff mixture.

The term “crosslinked” is defined for the present invention as the presence of covalent bonds between macromolecules (such as proteins and carbohydrates) within the food stuff mixture.

In one embodiment, the protein used in the food stuff mixture can in principle be derived from any possible source, i.e. any possible organism or cell, such as animals, plants, bacteria or fungi, or animal cells, plant cells or fungal cells, as long as it is edible. “Edible” is herein defined as fit for human consumption, hence palatable and non-poisonous. Optionally, the protein is not derived from animal slaughter or farming. Optionally, the protein is derived from (a) plant(s).

The protein may be a naturally occurring protein or a recombinantly produced protein. Naturally occurring proteins are preferred. The protein may be in the form of an isolated protein which had previously been purified or partially purified from a source organism, parts thereof, tissue or from cells and/or it can be in the form of plant material which is directly processed. The protein may also be in the form of a fermented product produced from plant material which contains proteins.

In one embodiment, the provided protein may have undergone an additional processing step which improves the final amount of crosslinks established within the food stuff mixture, accelerates the crosslinking process or improves the outcome of the directional freezing. Additional processing steps may include, but are not limited to, fermentation, particle size reduction, hydration, fractionation or heat or enzymatic treatment.

If the protein is derived from plants, it is possible to use plant material which contains high quantities of protein. Examples are parts of plants, like storage organs or seeds, which are known to be rich in proteins. Suitable are, in particular, proteins derived from leguminous plants, such as peas, lentils, beans etc. Examples for leguminous plants from which the protein present in the edible matrix can be derived are peas (e.g. yellow peas, chickpeas), beans (e.g. soy beans, kidney beans) and lentils. Other plants from which the protein containing material may be derived are cereals (such as rice, wheat, oat, rye), corn, oilseed (such as rapeseed, sunflower seeds and pumpkin seeds) and all sorts of vegetables. The protein can also be derived from seaweed or algae, such as Chlorella vulgaris. It is also possible to use protein from other single-cell organisms, such as yeast or from fungi.

The protein may also be a combination of several different proteins from the same or from different sources. Enzymes and protein types are combined to ensure that the enzyme can crosslink at least a fraction of the protein.

Optionally, the food stuff mixture comprises more than one distinctively different protein. The proteins may be mixed as dry powders before the addition of water.

In one embodiment, the food stuff mixture at least comprises pea protein, or a combination of pea protein and soy protein with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and chickpea protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and wheat protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and mung bean protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and fava bean protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and lupin protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises pea protein, or a combination of pea protein and potato protein, with at least 5 wt % of the comprised protein being pea protein, optionally at least 20 wt % being pea protein, optionally at least 40 wt % being pea protein, optionally at least 50 wt % being pea protein.

In one embodiment, the food stuff mixture at least comprises soy protein, or a combination of soy protein, mung bean protein and chickpea protein, wherein the soy protein is at least 20 wt % of the comprised protein, optionally at least 40 wt %, optionally at least 50 wt % and the mung bean protein at least 5 wt % of the comprised protein, optionally at least 15 wt %, optionally at least 25 wt %, with the residual wt % of the comprised protein being covered by the comprised chickpea protein. For example, the protein may be provided as part of a powder which is at least partly insoluble, optionally mostly insoluble, with insolubility being equal or more than 5%, optionally more than 10%, more than 20%, more than 30%, optionally more than 40%, more than 50% more than 60%, more than 80%.

“Insolubility” is defined according to the method by Peters et al. (Food Hydrocolloids Volume 65, April 2017, Pages 144-156) as the percentage of protein in a solution that sediments upon centrifugation at a predetermined speed and amount of time. “Protein” in this regard means the total amount of protein within the powder, which can be less than 100%, depending on the source of powder and manufacturer.

The powder(s) can further comprise carbohydrates, fats and salts depending on the source of powder and manufacturer.

Without wanting to be bound by theory, it has been found by the inventors that the average particle size of the powder has an influence on the sensory perception and obtained structure. It is believed that providing the protein as a powder with average particle sizes equal to or smaller than 300 μm may be advantageous in this respect. Optionally, the protein is provided as a powder with an average particle size smaller than or equal to 200 μm or smaller than 150 μm. In an embodiment, the average particle size is smaller than or equal to 50 μm. The “average particle size” is defined herein as the median particle diameter X₅₀, the volume mean diameter obtained according to the standards NF X11-666 or ISO 13320-20.

Optionally, the protein content in the food stuff mixture is at least 2 wt %, optionally at least 5 wt %, optionally at least 8 wt %, optionally at least 12 wt %, more than 20 wt % or more than 25 wt %.

The proteins may be partly pre-texturized in form of protein that previously underwent low moisture extrusion with a moisture content of less than 40 wt %, optionally followed by drying and/or rehydration. Alternatively, texturized proteins that previously underwent high moisture extrusion with a moisture content of more than 40%, optionally followed by drying, hydration, marination, rehydration, soaking or curing or other post-processing methods may be utilized.

Optionally, the food stuff mixture also contains carbohydrates. The amount of carbohydrates is optionally more than 0.1 wt. %. The carbohydrates can be added in the form of a powder or emulsion and can, for example, be selected from the group consisting of mono-, di-, oligo- and polysaccharides, e.g. complex polysaccharides, such as glucose, maltose, dextrose, mannose, alginate, alginate sulfate, starch, modified starch, gelatin, pectin, cellulose, bacterial cellulose, chitosan, agar, gellan gum, acylated and/or sulfated gellan gum, guar gum, cassia gum, konjac gum, arabic gum, ghatti gum, locust bean gum, xanthan gum, xanthan gum sulfate, carrageenan, carrageenan sulfate, and a combination or mixture of any of the above. Selected carbohydrates such as, for example pectin, can also take part in the crosslinking reaction depending on the crosslinking enzyme utilized (Wemmer J. et al., Food Funct. 2020; 11:2040-2047).

The food stuff mixture may also contain edible lipids i.e. oils or fats, optionally less than 10 wt %, optionally less than 5 wt %. Alternatively, the food stuff mixture may also contain optionally less than 30 wt %, optionally less than 25 wt % edible lipids. The lipids can be added in the form of a liquid, solid or in the form of an emulsion, with the emulsion used being an oil-in-water, water-in-oil or water-oil-water emulsion.

The food stuff mixture further comprises water in which all other components may be dispersed and/or partly dissolved. In one embodiment, the food stuff mixture at least comprises pea protein, or a combination of pea protein and mung bean protein, with at least 5 wt % pea protein, optionally at least 20 wt % pea protein, optionally at least 50 wt % pea protein, optionally at least 80 wt % pea protein.

The food stuff mixture may also comprise viable or non-viable animal cells derived from fermentation or in-vitro tissue culture, whereas these animal cells may be involved in crosslinking, are part of the protein-rich lamellar or fibrillar structure or just act as a filler.

Furthermore, heat sensitive nutrients may be added to the food stuff mixture. The heat sensitive nutrients can, for example, be selected from the group consisting of vitamins such as B-vitamins (folate and thiamin) and vitamins A, C, D and vitamin E.

The food stuff mixture may also contain living starter cultures such as bacteria, fungi, mycelium or spores. These live cultures may add to, consume, or transform protein and other comprised ingredients provided for the food stuff mixture before freezing.

The food stuff mixture may also contain vitamins, trace elements, flavor compounds, colorants, and/or salts. Optionally, at least some of the vitamins, flavor compounds or colorants are heat sensitive i.e. degrade or change structure or functionality upon heating above 50° C.

The pH of the food stuff mixture may be controlled before freezing using food grade organic or inorganic acids or bases. The pH is adjusted according to the protein(s) utilized in the food stuff mixture. In one preferred embodiment, the pH is adjusted to values between 4 and 9.

The food stuff mixture may be 3D printed on a flat metallic surface before freezing. In one configuration, the 3D printed food stuff mixture is surrounded on all sides but the metal side by an insulating material during freezing. More than one food stuff mixtures may be printed together from different print heads to create different phases of a single object, which is then frozen in a mold.

The food stuff mixture may be provided in the form of a foam or an emulsion rather than a dispersion of protein powder particles in water.

Two or more different food stuff mixtures may be provided in the same mold but with each mixture as a still clearly identifiable phase, and frozen together. The different phases can be arranged in a layered fashion or any other predefined pattern within the mold. The different phases can either originate from a predetermined pattern or from a phase separation event between two mixed phases. One of the two phases can also be supplied in a frozen state and placed within or next to the second phase.

The food stuff mixture contains a crosslinking enzyme which optionally requires temperatures of at least 10°, optionally of at least 4° C. to be active, optionally of at least 2° C. to be active, optionally the enzyme is still active at temperatures above 20° C. Optionally, at temperatures of 4° C., the crosslinking enzyme shows a relative activity of at least 1%, optionally more than 5%, optionally more than 10%. In certain instances, the enzymes are active within a temperature range of 4° C. to 50° C. The crosslinking enzyme can be in principle from any source and must be able to form covalent bonds between proteins, peptides or amino acids. The crosslinking enzyme can, for example, be selected from the group consisting of transglutaminases, sortases (Broguiere N. et al., Acta Biomat. 2018 Sep. 1; 77:182-190), lysyl oxidases and amine oxidases (Lucero, H. A., et al., Cell. Mol. Life Sci. 2006; 6:2304-2316), tyrosinases (Lantto R. et al. J Agric Food Chem. 2007 Feb. 21; 55 (4): 1248-55), laccases (Yamaguchi, S. (2000) Method for crosslinking protein by using enzyme. U.S. Pat. No. 6,121,013) and peroxidases (Stahmann M. A., Adv Exp Med Biol. 1977; 86B: 285-98). The covalent connections formed by these enzymes are ε-(γ-glutamyl) lysine bridges, an Leucine-Proline-X-Threonine-Glycine motif bridge (LPXTG motif, X can be any amino acid), Schiff-Base or aldol condensation products, o-quinone and dityrosyl-type crosslinks. The enzymes can, for example, be selected from the group consisting of tyrosinases, laccase or peroxidases, wherein the covalent connections formed by these enzymes are optionally in the form of o-quinones crosslinks in case of tyrosinases and dityrosyl-type crosslinks in case of laccase and peroxidases.

The food stuff mixture may also be pre-fermented using microorganisms before the addition of the crosslinking enzyme and before freezing.

The crosslinking enzyme may be transglutaminase from either animal or microbial origin with a specific enzyme activity of at least 90 Uₛ/g. Optionally, the crosslinking enzyme may be transglutaminase from microbial origin. The amount of transglutaminase in the food stuff mixture before freezing is at least 0.01 wt. % (0.9 U per 100 g of the comprised protein content), optionally at least 0.5 wt. % (45 U per 100 g of the comprised protein content), optionally at least 1 wt. % (90 U per 100 g of the comprised protein), optionally at least 1 wt. % (90 U per 100 g of the comprised protein), and/or not more than 6 wt. % (540 U per 100 g of the comprised protein content).

The food stuff mixture may further comprise spices, herbs, colorants, flavor, aroma components or other edible ingredients to tailor texture, taste, colour or other attributes.

In step (b) of the process according to the present invention, the food stuff mixture optionally is placed in a mold to be frozen as to create a frozen, texturized food stuff mixture.

The mold is not limited to any specific geometric shape as long as it can contain the food stuff mixture. The mold may partially or completely enclose the food stuff mixture and be made from one or several different materials. The mold surfaces which are directly exposed to the cooling medium, or the surfaces which have a higher heat conductivity than other surfaces, determine the thermal gradient and hence the orientation of the directional freezing in the food stuff mixture.

“Directional freezing” in the present invention is defined as the anisotropic freezing of a food stuff mixture, where elongated ice crystals form along a temperature gradient. Simultaneously, solids within the food stuff mixture are concentrated between the formed ice crystals.

The term “anisotropic”/“anisotropy” refers to the property of a material by which the material has different properties in different directions as opposed to isotropy. In the context of the present invention, it refers, in particular, to the formed ice crystals and the resulting lamella or fibers made from the solids within the food stuff mixture which have a preferred orientation, giving the food stuff mixture texture. Anisotropic ice crystals may be elongated or lamellar.

The food stuff mixture may be placed in a mold of any shape. At least one surface of the mold may be cooled. The cooled surface may be cooled by exposing it to a cooling medium (e.g., a liquid cooling medium), e.g. via one or more channels running through the mold. The mold may comprise at least one flat surface (e.g., a flat metallic surface) exposed to at least one cooling medium (e.g., a liquid cooling medium). Alternatively, two or more surfaces may be in contact with two or more different cooling media with different temperatures or different heat transfer coefficients, such that a thermal gradient is created within the food stuff mixture. Optionally, at least some or all other surfaces may be made from an insulating material (i.e., a material that is considered a suitable thermal insulator by those skilled in the art).

The food stuff mixture may be placed directly on or in contact with a cold surface (e.g., a cold metallic surface) exposed to a liquid cooling medium without a mold, e.g. in a way that the food stuff mixture does not come into contact with the liquid cooling medium. Alternatively, a mold optionally comprising a flat metallic surface containing the food stuff mixture may be placed on a cold metallic surface set to a predefined temperature between −196° C. and 0° C.

The liquid cooling medium may have a temperature between −196° C. and 0° C. It is possible that the liquid cooling medium is set to −15° C., optionally to −10° C., before placing the food stuff mixture into the mold.

The food stuff mixture may be placed in an insulated plastic mold with one open side where the food stuff mixture is directly exposed to a gaseous cooling medium. In an embodiment, the gaseous cooling medium is air. In an embodiment, the cool air is at −20° C.

The food stuff mixture may be cooled at a cooling rate of at least 0.01° C./min, but less than 100° C./min. Optionally, the cooling rate is at ranging between 0.01° C./min and 10° C./min, optionally between 0.05° C./min and 0.5° C./min. The term “cooling rate” in the present invention describes the reduction of the temperature per minute in the food stuff mixture at a distance of 0.5 cm to the cooling medium (where the food stuff mixture is directly exposed to the cooling medium) and/or at a distance of 0.5 cm to the cold surface (e.g., the cold metallic surface) of the mold (where a mold with such cold surface is used, as described above).

The food stuff mixture may be cooled below the freezing temperature of water down to a predefined temperature for at least 1 h or until the entire food stuff mixture or part of the food stuff mixture reaches the predefined temperature. The food stuff mixture may be frozen once more after thawing, e.g. after thawing for 24 h at a temperature of 4° C. According to the methods of the present invention, the directionally frozen, texturized food stuff mixture is placed at temperatures above the freezing point of water to melt the ice crystals (“thawing”), optionally at least 0° C., in the mixture and to activate the crosslinking enzyme and to crosslink the food stuff mixture. Several frozen food stuff mixtures can also be stacked on top of each other or placed next to each other in any order before thawing and crosslinking the frozen food stuff mixture.

The term “crosslinked” in light of the present invention is defined in that covalently bonded macromolecules within the texturized food stuff mixture form an at least partly percolated network. Crosslinking provides the texturized food stuff mixture with the ability to elastically respond to a mechanical input. The elastic response to mechanical input increases in comparison to before or without crosslinking.

The thawing of the texturized frozen food stuff mixture may be achieved by exposing the mold or the food stuff mixture directly to a medium which is warmer than the freezing point of water, where the medium may be a gas such as air or a liquid such as water.

The crosslinking with the enzyme may be achieved by placing the mold containing the texturized food stuff mixture or the texturized food stuff mixture with or without the mold in a water bath to heat the texturized food stuff mixture uniformly. Alternatively, the mold containing the texturized food stuff mixture or the texturized food stuff mixture with or without the mold may be placed in a chamber with a temperature above the freezing point of water, like a refrigerator, water bath or an oven.

The texturized food stuff mixture may be placed in a fridge as for the core of the food stuff to reach a temperature of 4° C. for at least 12 h. Optionally, the texturized food stuff mixture is heated for the core temperature to reach 50° C. for at least 30 min, wherein the core of texturized food stuff mixture is heated at a rate of less than 15° C./min.

The crosslinking enzyme may be deactivated through heating the crosslinked texturized food stuff above the stability temperature of the enzyme. Optionally, the deactivation temperature utilized is at least 60° C., but may depend on the enzyme type and origin, for at least 10 min, e.g. for transglutaminase.

The crosslinked texturized food stuff after crosslinking optionally comprises more than 60 wt % of the initial water content of the initial food stuff mixture, optionally more than 70 wt %, optionally more than 80 wt %, optionally more than 85 wt %, optionally more than 90 wt %. Meaning that the process does not lead to high water loss providing a water-rich and juicy food stuff.

Optionally, the crosslinked texturized food stuff comprises at least 40 wt % water, optionally at least 60 wt % water, optionally at least 75 wt % water.

Optionally, the crosslinked texturized food stuff comprises less or equal to 95 wt % of water, optionally less or equal to 85 wt % or less or equal to 80 wt %, optionally less or equal to 75 wt %.

Optionally, an elastic modulus in uniaxial compression (as described in “Texture in Food”, Volume 2, Woodhead Publishing Series in Food Science, Technology and Nutrition, 2004) of the crosslinked texturized food stuff mixture is at least 1 kPa, optionally at least 10 kPa. Optionally, the elastic modulus in uniaxial compression is in a range of 1 kPa to 5 MPa, optionally in a range of 10 kPa to 2 MPa. Such elastic modulus in uniaxial compression is similar to that of fish or meat.

The crosslinked texturized food stuff after crosslinking may display an elastic modulus in uniaxial compression, which is higher than such elastic modulus in uniaxial compression of a texturized food stuff mixture prepared by only freeze-structuring and thawing and without addition of a crosslinking enzyme.

In some embodiments, the stabilized, texturized food stuff is exposed to an acid, base or salts, either in solution or in solid form, to further stiffen, soften, colour or change the taste of the crosslinked texturized food stuff.

In some other embodiments, the stabilized, texturized foods stuff is further processed by incubating the whole, chunks, pieces or fibers of the stabilized, texturized food stuff in the presence of microorganisms. These microorganisms can be either bacteria, fungi or fungal spores, and can be added in the form of single cells or pluri-cellular assemblies. These microorganisms can be applied topically to the stabilized, texturized food stuff, injected into the food stuff via a syringe or be infused via a process like vacuum infiltration. Chunks or fibers prior or post-fermentation may be reassembled into whole pieces again, either through the interaction of the biomass deposited by the microorganisms or through a food-grade, edible adhesive. Optionally, a fungus or a combination of fungi is used. Optionally, at least one fungus is selected from the group comprising Zygomycota and Ascomycota, optionally from a genus selected from the group comprising Aspergillus, Rhizopus, Penicillium and Fusarium. Optionally, the at least one fungus belongs to a species selected from the group comprising Aspergillus oryzae, Rhizopus oligosporus, Penicillum camemberti and Fusarium venenatum.

In another embodiment, said at least one fungus is selected from the division of basidiomycetes, optionally from a genus selected from the group comprising Pleurotus, Laetiporus, Lentinula, Ganoderma, Grifola, Agaricus, Flammulina, Morchella, Hypholoma, Macrolepiota and Cantharellus. Optionally, the at least fungus belongs to a species selected from the group comprising Pleurotus sapidus, Pleurotus ostreatus, Pleurotus djamor, Laetiporus sulphureus, Lentinula edodes, Ganoderma lucidum, Grifola frondosa, Agaricus bisporus, Flammulina velutipes, Morchella angusticeps, Hypholoma capnoides, Macrolepiota procera and Cantharellus cibarius.

The crosslinked texturized food stuff may undergo further post-processing. For example, post processing can include cutting, pressing, rolling, pulling, flavoring, coloring, preserving, cooking, frying, marinating, dehydrating, rehydrating, marinating, confiting, packaging, soaking, spicing, salting, or smoking. The product may be used, further processed, packed and marketed like animal meat products.

Following the process presented in this invention, results in the formation of a crosslinked texturized food stuff.

The crosslinked texturized food stuff may be further soaked in a liquid to increase juiciness. This liquid may be water- or lipid-based and either warm or cold. For example, the crosslinked texturized food stuff may be soaked in liquid fat, which gets partly absorbed into the lamellar or fibrillar structure followed by solidification through cooling. Thus, layers of fat may be introduced into the structure.

The crosslinked texturized food stuff may be further partly dried by hot air drying, vacuum drying, freeze drying or other drying methods to reduce the water content.

The crosslinked texturized food stuff resulting from the methods according to the present invention comprises proteins, whereas at least some of the proteins are covalently crosslinked, and an anisotropic lamellar or fibrillar structure, optionally visible by human eye, and at least residues of an active or de-activated crosslinking enzyme. Optionally, the food stuff further comprises at least 40 wt % water.

The crosslinked texturized food stuff can exhibit a Young's modulus of at least 1 kPa, preferably at least 10 kPa, optionally at least 100 kPa in uniaxial compression.

The crosslinked texturized food stuff may have a volume of at least 1 cm³, optionally at least 4 cm³.

Optionally, said crosslinked texturized food stuff comprises a heat sensitive component, which has not been heated to above a critical temperature to degrade said heat sensitive component in the process of making said crosslinked texturized foodstuff.

The crosslinked texturized food stuff may further comprise spices, salts, acids, flavours, colorants, carbohydrates, lipids, herbs, dietary fibers or other food components to tailor texture, taste, appearance, nutritional content or colour.

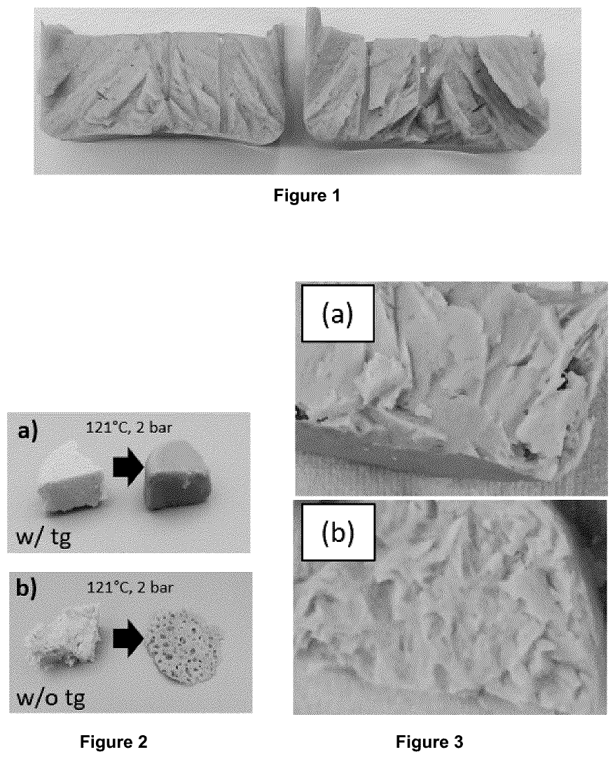

As shown in FIG. 1, an exemplary crosslinked texturized food stuff is composed of 25 wt % pea protein isolate powder and 0.25 wt. % transglutaminase (26.88±4.38 U per 100 g of food stuff mixture). When broken in half, the stabilized texture is clearly visible throughout the entire structure. Shown are the two halves after breaking a cylindrical shaped food stuff (8 cm diameter, 4 cm height).

For comparison, FIG. 2 depicts a crosslinked texturized food stuff composed of 25 wt % pea protein isolate powder and 0.25 wt. % transglutaminase (26.88±4.38 U per 100 g of food stuff mixture) (w/ tg) and texturized food stuff mixture composed of 25 wt % pea protein only (w/o tg) before and after autoclaving at 121° C. and 2 bar pressure. The sample without transglutaminase before autoclaving shows deformation due to its softness. After autoclaving, the food stuff with transglutaminase retained its shape, while the one without transglutaminase loses its shape as the pea protein isolate is poorly heat gelling.

FIG. 3 depicts a crosslinked texturized food stuff composed of 25 wt % pea protein isolate powder, 2 wt % pea fibers, 1 wt % rapeseed oil and 0.25 wt. % transglutaminase (26.88±4.38 U per 100 g of food stuff mixture). When broken in half, the stabilized texture is clearly visible throughout the entire structure. Shown are the two halves after breaking a cylindrical shape (8 cm diameter, 4 cm height) parallel to the short axis (a) as well as perpendicular to the short axis (b).

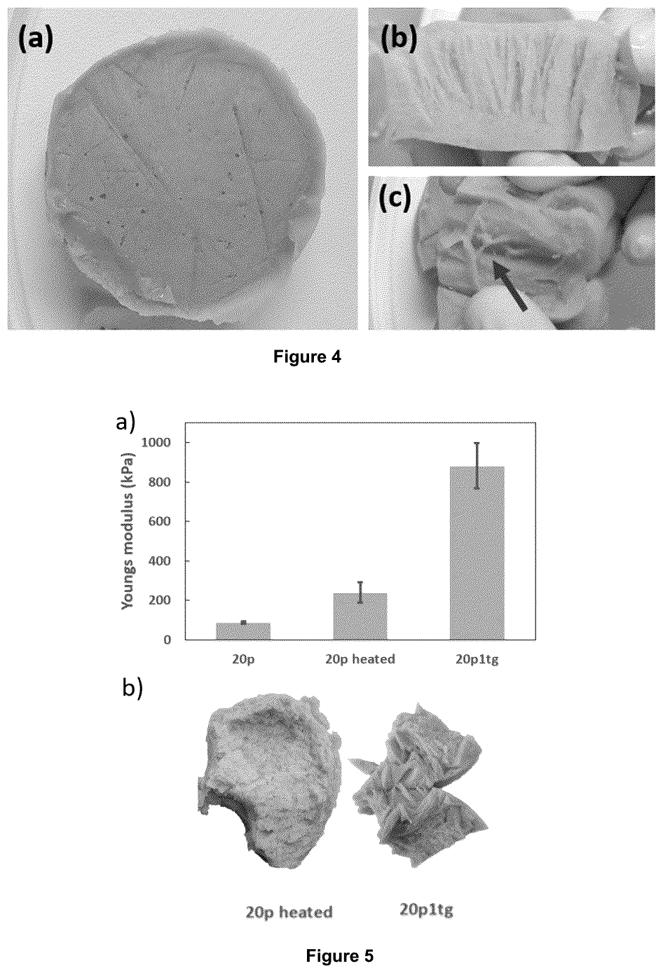

As can be seen in FIG. 4, a further exemplary crosslinked texturized food stuff is composed of 15 wt % pea protein isolate powder and 0.15 wt. % transglutaminase (16.13±2.63 U per 100 g of food stuff mixture). (a) shows a top view of the stabilized food stuff mixture directly after the stabilization process with the frozen structure clearly visible. (b) shows a side view after breaking the cylindrical shape shown in (a) parallel to the short axis in half. The obtained lamellae are finer than in FIG. 1. When pulling apart, fine interlamellar fibers are visible as indicated by the arrow in (c).

FIG. 5 shows a crosslinked texturized food stuff composed of 25 wt % pea protein isolate powder which was either not crosslinked (20p), heat treated (20p heated) according to Example 6 or enzymatically crosslinked with 0.25 wt. % transglutaminase (26.88±4.38 U per 100 g of food stuff mixture) (20p1tg). (a) shows the different Young's moduli achieved with the two different crosslinking methods compared to not crosslinked food stuff mixture. (b) shows the resulting structures found when looking into the food stuff mixture either after heat treatment (20p heated) or enzymatic treatment (20p1tg).

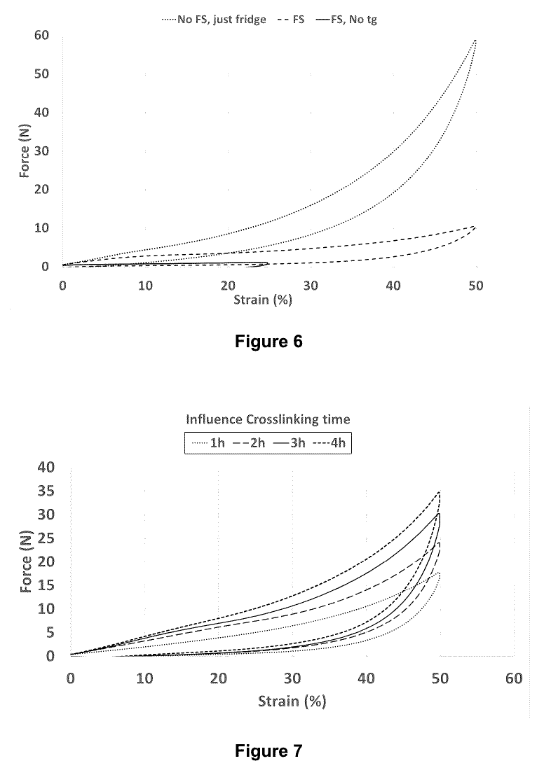

The difference between crosslinking and freeze structuring and crosslinking (FS) along without the process of freeze structuring (no FS, just Fridge) can be seen in FIG. 6 with samples produced according to Example 7. It clearly indicates how the structuring process influences the mechanical properties in uniaxial compression of the final crosslinked food stuff mixture and how a simple structuring process without the crosslinking results in a soft, pasty non-elastic structure (FS, no tg).

The mechanical properties can also be tuned by altering the crosslinking time at 50° C. as shown in FIG. 7, which depicts the mechanical response of texturized food stuff exposed to uniaxial compression crosslinked for different amounts of time according to Example 8.

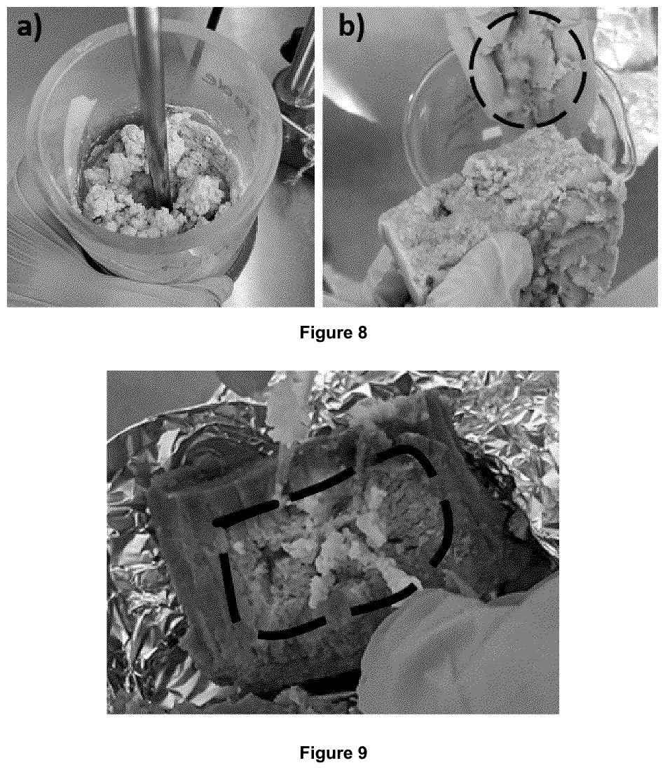

FIG. 8a shows that adding chemical crosslinkers such as glutaraldehyde before directional freezing leads to premature crosslinking during preparation of the food stuff mixture according to Example 9. The resulting structure after attempted directional freezing is shown in FIG. 8b. No fibrils, lamella or similar structures are visible when breaking the structure apart.

Crosslinking the texturized, frozen food stuff using chemical crosslinkers such as glutaraldehyde after directional freezing according to example 10 leads to a partly texturized food stuff, where only the outermost material of the food stuff is crosslinked and texturized, whereas the center of the food stuff does not contain any textured or crosslinked food stuff as indicated in the marked area in FIG. 9.

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

As used herein the term “about” means approximately, in the region of, roughly, or around. When the term “about” is used in conjunction with a numerical value or range, it modifies that value or range by extending the boundaries above and below the numerical value(s) set forth. In general, the term “about” is used herein to modify (a) numerical value(s) above and below the stated value(s) by 10%.

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLES

The following examples are provided for carrying out the invention in accordance with the claims.

### Example 1

25 wt % pea protein isolate powder, resulting in a protein content of 20 wt %, 0.25 wt. % transglutaminase (active between 2-60° C., 26.88±4.38 U per 100 g of food stuff mixture), corresponding to 134.42±21.92 U transglutaminase per 100 g of the protein content, was dispersed in tap water filled up to 100 wt % and mixed in a 400 ml plastic beaker using an overhead stirrer at room temperature until a homogeneous mixture was obtained. Within maximum 30 min after mixing, the beaker with the food stuff mixture was then placed in a styrofoam container, enclosing the beaker on all but the open side. This setup was then placed in a walk-in freezer at −20° C., cooling at a rate between 0.015° C./min and 0.5° C./min for a minimum of 10 h. Afterwards, the beaker with the frozen food stuff mixture was removed from the freezer and placed in a water bath (25° C.) for 4 h. From this, a crosslinked texturized food stuff was obtained as shown in FIG. 1.

### Example 2

25 wt % pea protein isolate powder (same protein isolate powder as in Example 1) with or without 0.25 wt. % transglutaminase (active between 2-60° C., 26.88±4.38 U per 100 g of food stuff mixture), corresponding to 134.42±21.92 U transglutaminase per 100 g of the protein content, was dispersed in tap water and mixed in a 400 ml plastic beaker using an overhead stirrer until a homogeneous mixture was obtained. The beaker with the food stuff mixture was then placed in a styrofoam container, enclosing the beaker on all but the open side. This setup was then placed in a walk-in freezer at −20° C., cooling at a rate between 0.015° C./min and 0.5° C./min for a minimum of 24 h. Afterwards, the beaker with the frozen food stuff mixture was removed from the freezer and placed in a water bath (25° C.) for 4 h. From the sample with transglutaminase, a crosslinked texturized food stuff was obtained whereas in the case of the food stuff mixture without transglutaminase a soft, structured food stuff was obtained that would yield upon application of a mechanical force. Circular plugs were punched out from the food stuff mixtures (but any shape is possible) and placed separately in an autoclave at 121° C. and 2 bar pressure for 20 min to further gel the food stuff. The difference between the sample with or without transglutaminase is shown in FIG. 2.

### Example 3

15 wt % pea protein isolate powder (same protein isolate powder as in Example 1) and 0.15 wt. % transglutaminase (active between 2-60° C., 16.13±2.63 U per 100 g of food stuff mixture), corresponding to 134.42±21.92 U transglutaminase per 100 g of the protein content, were dispersed in tap water and mixed in a 400 ml plastic beaker using an overhead stirrer until a homogeneous mixture was obtained. The beaker with the food stuff mixture was then placed in a styrofoam container, enclosing the beaker on all but the open side. Thus, the beaker with the food stuff mixture was insulated with styrofoam on all but one surface. The food stuff mixture was frozen in a cooling bath at −15° C., cooling at a rate between 0.05° C./min and 0.5° C./min for at least 24 h. After this, the beaker with the food stuff mixture was removed from the cooling bath and placed in a water bath at 25° C. for 4 h, respectively.

### Example 4

25 wt % pea protein isolate powder (same protein isolate powder as in Example 1), 2 wt % pea fibers, 1 wt % rapeseed oil and 0.25 wt. % transglutaminase (active between 2-60° C., 26.88±4.38 U per 100 g of food stuff mixture), corresponding to 134.42±21.92 U U transglutaminase per 100 g of the protein content, were dispersed in tap water and mixed in a 400 ml plastic beaker using an overhead stirrer until a homogeneous mixture was obtained. The beaker with the food stuff mixture was then placed in a styrofoam container, enclosing the beaker on all but the open side. This setup was then placed in a walk-in freezer at −20° C., cooling at a rate between 0.015° C./min and 0.5° C./min for a minimum of 10 h. Afterwards, the beaker with the frozen food stuff mixture was removed from the freezer and placed in a water bath (25° C.) for 4 h. From this, a crosslinked texturized food stuff was obtained as shown in FIG. 3.

### Example 5

15 wt % pea protein isolate powder (same protein isolate powder as in Example 1) and 0.15 wt. % transglutaminase (active between 2-60° C., 16.13±2.63 U per 100 g of food stuff mixture), corresponding to 134.42±21.92 U transglutaminase per 100 g of the protein content, were dispersed in tap water and mixed in a 400 ml plastic beaker using an overhead stirrer until a homogeneous mixture was obtained. This beaker with the food stuff mixture was insulated with styrofoam on all but one surface which was in contact with a cooling bath at −15° C., cooling at a rate between 0.05° C./min and 0.5° C./min for at least 24 h. After this, the beaker with the food stuff mixture was removed from the cooling bath and placed at 4° C. for 24 h, after which it was placed back in the cooling bath at −15° C. for 24 h. After this time period, the frozen food stuff mixture was removed from the cooling bath and placed in a water bath at 50° C. for 7 h. From this, a crosslinked texturized food stuff was obtained, shown in FIG. 4.

### Example 6

25 wt % pea protein isolate (same protein isolate powder as in Example 1) alone or in combination with 0.25 wt. % transglutaminase (active between 2-60° C., 26.88±4.38 U per 100 g of food stuff mixture), corresponding to 134.42±21.92 U transglutaminase per 100 g of the protein content, were dispersed in tap water and mixed in a 400 ml plastic beaker using an overhead stirrer until a homogeneous mixture was obtained. The beaker with the food stuff mixture was then placed in a styrofoam container, enclosing the beaker on all but the open side. Thus, the beaker with the food stuff mixture was insulated with styrofoam on all but one surface. The food stuff mixture was then frozen in a walk-in freezer at −20° C., cooling at a rate between 0.015° C./min and 0.5° C./min (measured at 0.5 cm distance from the cooling media) for at least 24 h. After this, the beaker with the food stuff mixture containing the transglutaminase was removed from the freezer and placed at 25° C. for 24 h. The food stuff mixture containing only the pea protein isolate was either placed at 25° C. for 24 h or placed at 85° C. for 2 h for heat treatment of the food stuff mixture. The mechanical properties of the resulting food stuff mixtures and the resulting structures are shown in FIG. 5, with the food stuff in FIG. 5a (20p heated) not displaying any lamellar structure when compared with the foods stuff in FIG. 5b (20p1tg) At the same time, 20p heated displays a much lower Young's modulus in comparison to 20p1tg.

### Example 7

22.5 wt % pea protein isolate powder (same protein isolate powder as in Example 1) with or without the addition of 0.225 wt. % transglutaminase (active between 2-60° C., 24.19±3.94 U per 100 g of food stuff mixture), corresponding to 134.39±21.88 U transglutaminase per 100 g of the protein content, was dispersed in cold tap water and mixed using an overhead stirrer in a 400 ml plastic beaker with a metal bottom until a homogeneous mixture was obtained. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. For comparison, the same food stuff mixture was alternatively placed at 4° C. in a fridge for 24 h. After this, the beakers with the food stuff mixture were removed from the metal surface or the fridge and placed at 50° C. for 4 h, followed by a pasteurization step at 90° C. for at least 30 min. After this time period, the texturized food stuff mixture was removed from the beaker and subjected to texture profile analysis using either 25% or 50% total strain. The different mechanical response from crosslinked texturized food stuff (FS), crosslinked non-texturized food stuff (no FS, just fridge) and non-crosslinked texturized food stuff (FS, No tg) are shown in FIG. 6.

### Example 8

22.5 wt % pea protein isolate powder (same protein isolate powder as in Example 1) with the addition of 0.225 wt. % transglutaminase (active between 2-60° C., 24.19±3.94 U per 100 g of food stuff mixture), corresponding to 134.39±21.88 U transglutaminase per 100 g of the protein content, was dispersed in cold tap water and mixed in a 400 ml plastic beaker with a metal bottom using an overhead stirrer until a homogeneous mixture was obtained. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. After this, the beaker with the food stuff mixture was removed from the metal surface and placed at 50° C. for either 1 h, 2 h, 3 h or 4 h, followed by a pasteurization step at 90° C. for at least 30 min. After this time period, the texturized food stuff mixture was removed from the beaker and subjected to texture profile analysis using 50% total strain. The different mechanical responses from crosslinked texturized food stuff after 1 h, 2 h, 3 h or 4 h crosslinking at 50° C. are shown in FIG. 7.

### Counter Example 9

25 wt % pea protein isolate powder (same protein isolate powder as in Example 1) was dispersed in a cold aqueous solution containing 1 wt % Glutaraldehyde in a 400 ml plastic beaker with a metal bottom using an overhead stirrer, ultimately resulting in a crumbly mixture due to the action of the glutaraldehyde. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. After this, the beaker with the food stuff mixture was removed from the metal surface and placed at 50° C. for 4 h, followed by a pasteurization step at 90° C. for at least 30 min. After this time period, the texturized food stuff mixture was removed from the beaker and the resulting internal structure visually inspected. The result is shown in FIG. 8.

### Counter Example 10

25 wt % pea protein isolate powder (same protein isolate powder as in Example 1) was dispersed in a cold water in a 400 ml plastic beaker with a metal bottom using an overhead stirrer until a homogeneous mixture was obtained. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. After this, the beaker with the food stuff mixture was removed from the metal surface and the still frozen food stuff mixture was immersed for 8 hours in a cold aqueous solution containing 10% Glutaraldehyde. The food stuff mixture was then removed from the solution and the resulting internal structure visually inspected. The result is shown in FIG. 9.

### Example 11

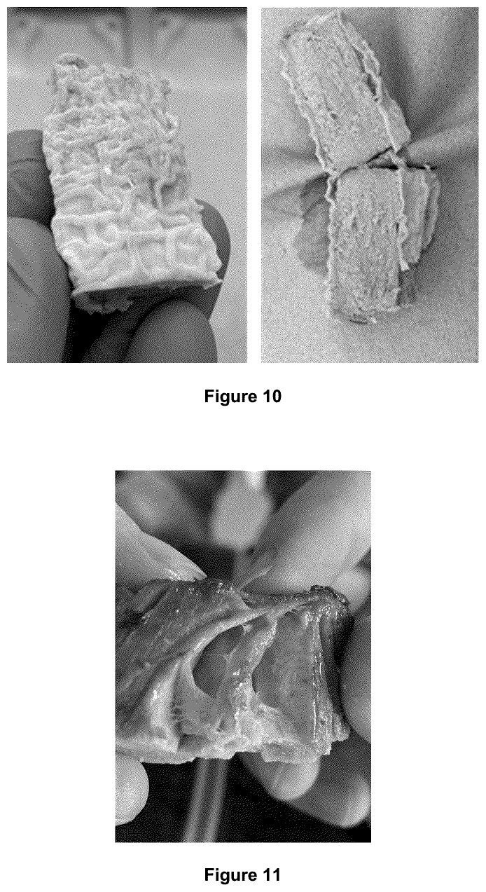

22.5 wt % pea protein isolate powder (same protein isolate powder as in Example 1) with the addition of 0.225 wt. % transglutaminase (active between 2-60° C., 24.19±3.94 U per 100 g of food stuff mixture), corresponding to 134.39±21.88 U transglutaminase per 100 g of the protein content, and 3% maize starch was dispersed in cold tap water and mixed in a 400 ml plastic beaker with a metal bottom using an overhead stirrer until a homogeneous mixture was obtained. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. After this, the beakers with the food stuff mixture were removed from the metal surface and placed at 50° C. for 4 h, followed by a pasteurization step at 90° C. for at least 30 min. After this time period, the texturized food stuff mixture was removed from the beaker and spores of Rhizopus oryzae were uniformly distributed to the entirety of the external surface of the structure at a concentration of ca. 10×10\hat{ }7 spores per 150 g of structure and fermented at 30° C. for 40 h in a closed box (permeable to oxygen and CO₂). The result is shown in FIG. 10.

### Example 12

22.5 wt % pea protein isolate powder (same protein isolate powder as in Example 1) with the addition of 0.225 wt. % transglutaminase (active between 2-60° C., 24.19±3.94 U per 100 g of food stuff mixture), corresponding to 134.39±21.88 U transglutaminase per 100 g of the protein content, was dispersed in cold tap water and mixed in a 400 ml plastic beaker with a metal bottom using an overhead stirrer until a homogeneous mixture was obtained. A mycelial biofilm produced by growing Rhizopus oryzae mycelium was then placed in the homogeneous mixture. This beaker with the food stuff mixture was placed on a metal surface and insulated by a polyethylene container. The metal surface was in contact with a cooling bath at −10° C., cooling at a rate between 0.05° C./min and 0.5° C./min for at least 24 h. After this, the beakers with the food stuff mixture were removed from the metal surface and placed at 50° C. for 4 h, followed by a pasteurization step at 90° C. for at least 30 min. After this time period, the texturized food stuff mixture was removed from the beaker and the resulting structure was pan fried and the structure opened by hand to show the connection of the mycelium and the fibers resulting from the texturizing process. The result is shown in FIG. 11.

The invention relates, in particular, to the any of the following aspects:
1. A method for preparing a crosslinked texturized food stuff, comprising the steps of:
   a) providing a food stuff mixture comprising at least a protein, water and a crosslinking enzyme;
   b) directionally freezing said food stuff mixture by cooling to below the freezing temperature of the food stuff mixture according to a directional thermal gradient such that ice crystals are grown anisotropically within the food stuff mixture to create a frozen texturized food stuff mixture;
   c) increasing the temperature of said frozen texturized food stuff mixture to a temperature at which the crosslinking enzyme is active and allowing the texturized food stuff mixture to form a crosslinked texturized food stuff.
2. The method according to aspect 1, where the protein used is at least partly soluble in water.
3. The method according to aspect 1 or 2, where the protein is supplied in the form of a powder, with an average particle size ranging from 0.1-200 μm, optionally from 1-50 μm.
4. The method according to any one of the preceding aspects, wherein the protein is provided in the food stuff mixture at a concentration of at least 2 wt %, optionally at least 5 wt %, optionally at least 8 wt %, optionally at least 12 wt %.
5. The method of any one of the preceding aspects, wherein said protein is not derived from animal slaughter or animal farming, optionally said protein is derived from plants, fungi, algae, bacteria or cell cultures.
6. The method of any one of the preceding aspects, wherein the food stuff mixture further comprises at least one of the substances selected from the group of lipids, carbohydrates, vitamins, minerals and trace elements.
7. The method of any one of the preceding aspects, wherein the food stuff mixture further comprises microorganisms, selected from the group of bacteria, fungi or fungal spores.
8. The method of any one of the preceding aspects, wherein the pH of the food stuff mixture is adjusted between 4 and 9, optionally wherein the pH adjustment is performed through the addition of food grade acids and bases.
9. The method of any one of the preceding aspects, wherein the crosslinking enzyme is selected from the group consisting of transglutaminase, sortases, lysyl oxidases and amine oxidases, tyrosinases, laccase or peroxidases, forming ε-(γ-glutamyl) lysine bridges, an LPXTG motif bridge or Schiff-Base or aldol condensation products, o-quinone or dityrosyl-type crosslinks, respectively.
10. The method according to any one of the preceding aspects, wherein the crosslinking enzyme is added in the food stuff mixture at a concentration relative to the comprised protein of at least 0.1 wt. % (9 U/100 g protein) optionally at least 0.5 wt. % (45 U/100 g protein), optionally at least 1 wt. % (90 U/100 g) protein, and/or wherein the crosslinking enzyme is added in the food stuff mixture at a concentration relative to the comprised protein of 5 wt. % (450 U/100 g protein) or less, optionally 2.5 wt % (225 U/100 g protein) or less.
11. The method according to any one of the preceding aspects, wherein the crosslinking enzyme is immobilized on or within a carrier, optionally wherein the carrier is maltodextrin.
12. The method according to any one of the preceding aspects, wherein the crosslinking enzyme is active from temperatures of at least 10° C., optionally at least 4° C., optionally at least 2° C. and wherein said crosslinking enzyme is optionally still active above 20° C.
13. The method according to any one of the preceding aspects, wherein the crosslinking enzyme is active at or below 70° C., optionally at or below 60° C.
14. The method according to any one of the preceding aspects, wherein step c) is carried out for at least 5 min, optionally at least 10 min, more preferably at least 20 min, optionally at least 30 min.
15. The method according to any one of the preceding aspects, wherein the method steps b) and c) are repeated at least once.
16. The method according to any one of the preceding aspects, wherein the food stuff mixture is shaped into a predefined form through casting, pressing, extrusion or 3D printing.
17. The method according to any one of the preceding aspects, wherein the directional freezing occurs from at least one distinct surface of the food stuff mixture (e.g., a surface exposed to surrounding cooling medium or to a cold surface of a mold).
18. The method according to any one of the preceding aspects, wherein the directional freezing is performed at temperatures within the range of 0° C. to −80° C., optionally within the range of −4° C. to −30° C., optionally within the range of −10° to −20° C.
19. The method according to any one of the preceding aspects, wherein the directional freezing is performed by providing the food stuff mixture in a container placed in a liquid cooling bath or a freezer.
20. The method according to any one of the preceding aspects, wherein in step c) the temperature is kept at a temperature above the melting point and below the deactivation temperature of the enzyme, optionally at a temperature at which at least 50% of the maximum enzyme activity is reached.
21. The method according to any one of the preceding aspects, wherein the method further comprises a step of
   d) deactivating the crosslinking enzyme, optionally by heating the crosslinked texturized food stuff to at least the deactivation temperature of the crosslinking enzyme.
22. The method of any one of the preceding aspects, wherein the method further comprises a step of
   e) pressing the crosslinked texturized food stuff to remove water and/or to increase the protein concentration.
23. The method of any one of the preceding aspects, wherein the method further comprises a step of
   f) fermenting whole or partial pieces of the crosslinked texturized food stuff using bacteria and/or fungi, optionally wherein at least one fungus is selected from the group consisting of ascomycetes, basidiomycetes, deuteromycetes, oomycetes, and/or zygomycetes.
24. The method of any one of the preceding aspects wherein the crosslinked texturized food stuff product is subjected to a further step selected from one or more of marinating, flavoring, tenderizing, dehydrating, pressing, hydrating, grilling, frying, boiling, smoking, or combinations thereof performed either together or in sequence, optionally followed by a step of
   g) cutting or slicing the crosslinked texturized food stuff, optionally along or perpendicular to the fiber direction.
25. A crosslinked texturized food stuff resulting from the method of any one of the preceding aspects comprising protein, water and at least residues of a crosslinking enzyme.
26. The crosslinked texturized food stuff of aspect 25, wherein at least some of the protein is crosslinked, comprising at least one ε-(γ-glutamyl) lysine bridge, or one LPXTG motif bridge or one Schiff-Base or one aldol condensation product or o-quinone or dityrosyl-type crosslinks.
27. The crosslinked texturized food stuff of aspect 25 or 26, wherein said food stuff has a lamellar or fibrillar structure.
28. The crosslinked texturized food stuff according to any of aspects 25 to 27, wherein said food stuff has a Young's modulus of at least 1 kPa measured in uniaxial compression.
29. The crosslinked texturized food stuff according to any of aspects 25 to 28, wherein the crosslinked texturized food stuff has in at least one dimension a height of at least 1 cm, optionally at least 2 cm and/or an area in cross-section of at least 15 cm², optionally at least 40 cm², optionally in the shape of the desired final product, optionally wherein the height is measured in a direction perpendicular to the plane of the cross-section.
30. The crosslinked texturized food stuff according to any of aspects 25 to 29, wherein the crosslinked texturized food stuff has a largest cross-sectional area of at least 40 cm².
31. The crosslinked texturized food stuff according to any one of aspects 25 to 30, wherein the crosslinked texturized food stuff comprises at least 2 wt %, optionally at least 5 wt %, optionally at least 8 wt %, optionally at least 12 wt % protein content.
32. The crosslinked texturized food stuff of any one of aspects 25 to 31, wherein the proteins are not derived from animal agriculture or slaughter, optionally wherein the proteins are derived from plant, single cell, algae or fungal origin or are derived from fermentation.
33. The crosslinked texturized food stuff according to any one of aspects 25 to 32, wherein the product further comprises lipids derived from plant, single cell, algae or fungal origin or derived from fermentation.
34. The crosslinked texturized food stuff of any one of aspects 25 to 33, wherein the product further comprises fibers derived from plant, single cell, algae or fungal origin or derived from fermentation.
35. The crosslinked texturized food stuff of any one of aspects 25 to 34, where the shape of the product has been obtained through means of casting, injection, pressing or 3D printing.
36. The crosslinked texturized food stuff of any one of aspects 25 to 35, wherein the crosslinked texturized food stuff has a water content of at least 80% of the water content of the food stuff mixture, optionally more than 90%, optionally more than 95%.
37. The crosslinked texturized food stuff of any one of aspects 25 to 36, wherein the crosslinked texturized food stuff has an elastic modulus between 1 kPa and 2 MPa measured in uniaxial compression, optionally between 10 kPa and 1 MPa measured in uniaxial compression.
38. The crosslinked texturized food stuff of any one of aspects 25 to 37, wherein the crosslinked texturized food stuff comprises at least 40 wt % water, optionally at least 60 wt % water, optionally at least 75 wt % water.
39. The crosslinked texturized food stuff of any one of aspects 25 to 38, wherein the food stuff is made by the method according to any one of aspects 1 to 24.
40. A food product comprising the crosslinked texturized food stuff of any one of aspects 25 to 39.

1. A method for preparing a crosslinked texturized food stuff, comprising steps of:
a) providing a food stuff mixture comprising at least a protein, water and a crosslinking enzyme;
b) directionally freezing said food stuff mixture by cooling to below a freezing temperature of the food stuff mixture according to a directional thermal gradient such that ice crystals are grown anisotropically within the food stuff mixture to create a frozen texturized food stuff mixture;
c) increasing a temperature of said frozen texturized food stuff mixture to a temperature at which the crosslinking enzyme is active and allowing the texturized food stuff mixture to form a crosslinked texturized food stuff.

2. The method according to claim 1, wherein the protein is provided in the food stuff mixture at a concentration of at least 12 wt %.

3. The method according to claim 1, wherein said protein is not derived from animal slaughter or animal farming.

4. The method of claim 1, wherein the food stuff mixture further comprises microorganisms, selected from the group of bacteria, fungi or fungal spores.

5. The method of claim 1, wherein the pH of the food stuff mixture is adjusted between 4 and 9.

6. The method of claim 1, wherein the crosslinking enzyme is selected from the group consisting of transglutaminase, sortases, lysyl oxidases and amine oxidases, tyrosinases or laccases or peroxidases forming ε-(γ-glutamyl) lysine bridges, an LPXTG motif bridge, a Schiff-Base or aldol condensation products or o-quinone or dityrosyl-type crosslinks, respectively.

7. The method according to claim 1, wherein the crosslinking enzyme is added in the food stuff mixture at a concentration relative to the protein of at least 0.1 wt. % (9 U/100 g protein) and up to 5 wt. % (450 U/100 g protein).

8. The method according to claim 1,
wherein the crosslinking enzyme is active from temperatures of at least 2° C. or
wherein the crosslinking enzyme is active at or below 70° C.

9. The method according to claim 1,
wherein step c) is carried out for at least 5 min.

10. The method according to claim 1,
wherein in step c) the temperature is kept at a temperature above a melting point of a frozen liquid contained within the frozen, texturized food stuff mixture and below a deactivation temperature of the enzyme.

11. The method according to claim 1,
wherein the steps b) and c) are repeated at least once.

12. The method according to claim 1, wherein the food stuff mixture is shaped into a predefined form through casting, pressing, extrusion or 3D printing.

13. The method according to claim 1,
wherein the step b) is performed at temperatures within a range of −4° C. to −30° C.

14. The method according to claim 1, wherein the method further comprises one or more of the following steps:
d) deactivating the crosslinking enzyme;
e) pressing the crosslinked texturized food stuff to remove water or to increase a protein concentration;
f) fermenting whole or partial pieces of the crosslinked texturized food stuff using bacteria or fungi;
g) cutting or slicing the crosslinked texturized food stuff.

15. A crosslinked texturized food stuff resulting from the method of claim 1, the crosslinked texturized food stuff comprising protein, water and at least residues of the crosslinking enzyme.

16. The crosslinked texturized food stuff of claim 15, wherein at least some of the protein is crosslinked, comprising at least one ε-(γ-glutamyl) lysine bridge, or one LPXTG motif bridge or one Schiff-Base or one aldol condensation product or o-quinone or dityrosyl-type crosslinks.

17. The crosslinked texturized food stuff of claim 15, wherein said crosslinked texturized foodstuff has a lamellar or fibrillar structure and a Young's modulus of at least 1 kPa measured in uniaxial compression.

18. (canceled)

19. The crosslinked texturized food stuff according to claim 15, wherein the crosslinked texturized food stuff further comprises lipids or fibers from a plant, fungi, single cell or fermented source.

20. The crosslinked texturized food stuff of claim 15, wherein the crosslinked texturized food stuff comprises at least 60 wt % water.

21. The crosslinked texturized food stuff of claim 15, wherein the crosslinked texturized food stuff has an elastic modulus between 10 kPa and 2 MPa measured in uniaxial compression.

22. A food product comprising the crosslinked texturized food stuff of claim 15.
